(19) United States
(12) Plant Patent Application Publication
(10) Pub. No.: US 2016/0073566 P1
(43) Pub. Date: Mar. 10, 2016

(54) Cannabis plant named 'AVIDEKEL'

(50) ;

(71) Applicant: Ytzchak COHEN, Tel Aviv (IL)

(72) Inventor: Ytzchak COHEN, Tel Aviv (IL)

(21) Appl. No.: 14/757,039

(22) Filed: Nov. 12, 2015

Related U.S. Application Data
(63) Continuation of application No. 14/193,252, filed on Feb. 28, 2014.
(60) Provisional application No. 61/768,567, filed on Feb. 25, 2013.

(51) Int. Cl. A01H 5/00

(52) U.S. Cl. PLT/258

(57) ABSTRACT

The disclosure relates to a new and distinct cultivar of Cannabis Sativa plant named ‘Avidekel’, characterized by a high amount of Cannabidiol (CBD) (16.3%) and a very low amount of Tetrahydrocannabinol (THC, 0.8%).

### BACKGROUND

The disclosure is directed to a new and distinct cultivar of Cannabis Sativa plant named ‘Avidekel’, characterized by a high amount of Cannabidiol (CBD) (16.3%) and a very low amount of Tetrahydrocannabinol (THC, 0.8%).

### SUMMARY

Provided herein is a new and distinct Cannabis Sativa L ssp. indica plant named Avidekel, characterized by a high amount of Cannabidiol (CBD) of greater than approximately 16% and a very low amount of Tetrahydrocannabinol (THC) of less than 1%, as illustrated and described herein.

### BRIEF DESCRIPTION OF THE DRAWINGS

FIG. 1 illustrates a single fully grown plant of the variety;

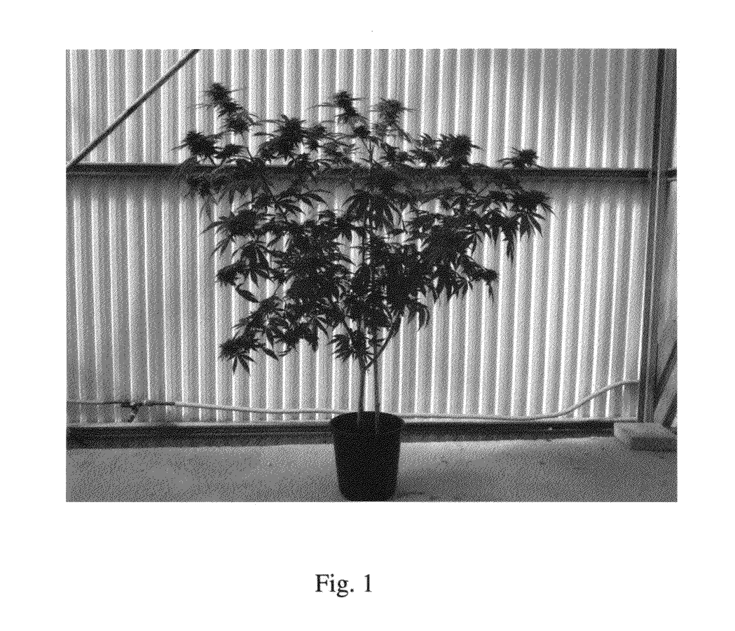

FIG. 2 illustrates a single leaf of the variety;

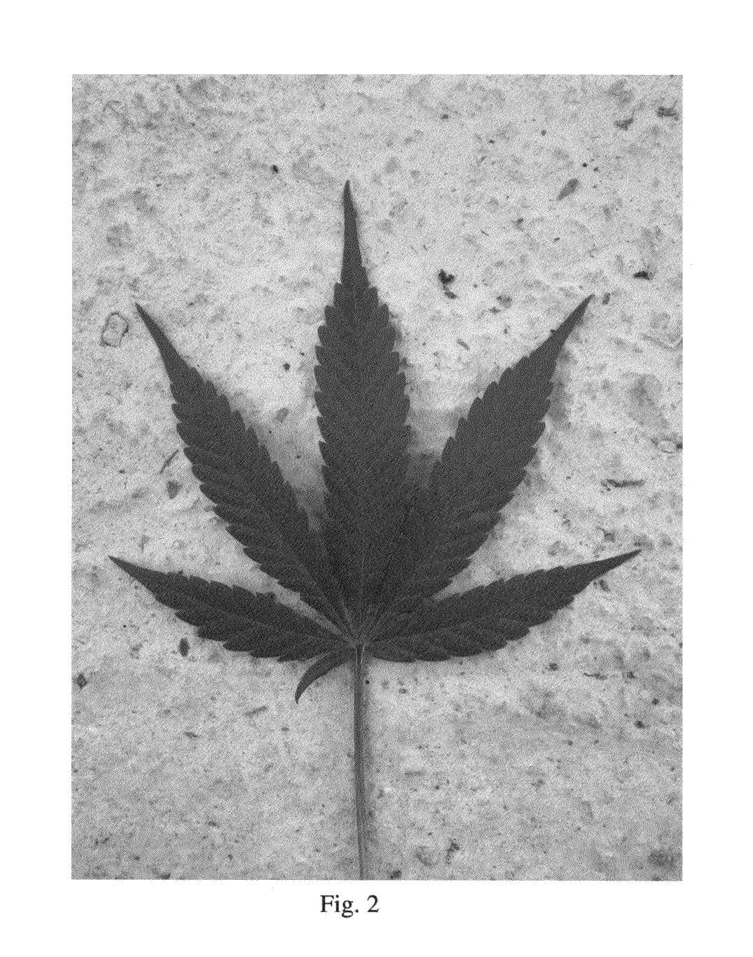

FIG. 3 illustrates a flowering plant in close-up; and

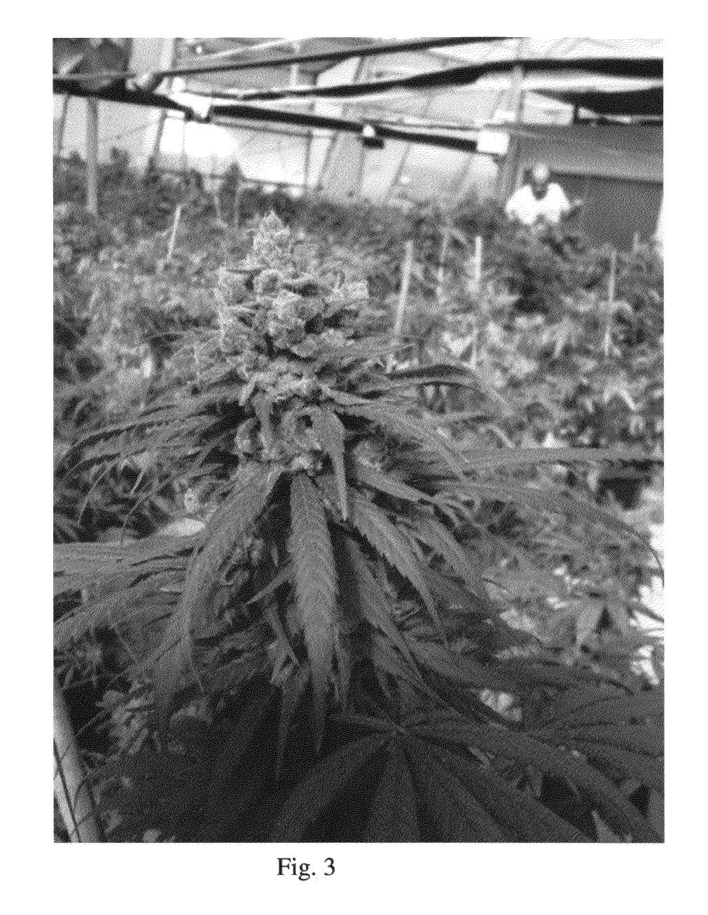

FIG. 4 illustrates a dried bud.

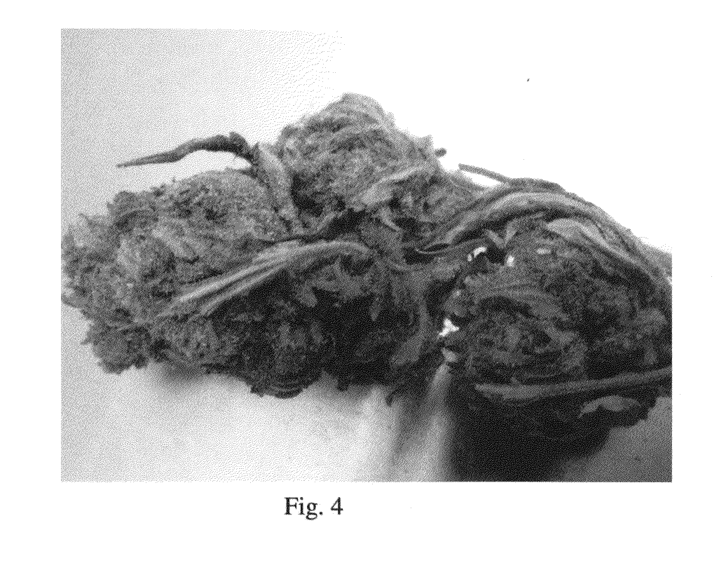

### DESCRIPTION

This invention relates to a new hybrid marijuana plant, Cannabis sativa L. ssp. indica named Avidekel. The Avidekel is a medicinal variety of marijuana notable for a high amount of Cannabidiol (CBD) (16.3%) and a very low amount of THC (0.8%).

The present plant was developed over 3 years through selective breeding from parents of unknown provenance in Birya, Israel. Generally production occurs through asexual propagation using stem cuttings. The plant may be grown outside in a normal warm summer, including many locations below 50N and virtually all climates below 45N.

### PLANT

### Exposed Plant Structure

The Plant is an annual, dioecious plant with bush-like stature. The natural height at 5 months old of indoor growth is −130 cm. A detailed list of characteristics follows:
Botanical classification: Cannabis sativa L. ssp. indica
Parentage unknown
Propagation: Stem cuttings.
Time to initiate roots in summer:
   Plant description.—annual, dioecious flowering shrub; multi-stemmed; freely branching; removal of the terminal bud enhances lateral branch development.
   Root description.—Short taproot (less than 30 cm). Medium to fine, dense lateral roots; white in color, brownish when “potbound” (rootbound).
First year stems:
   Shape.—round. Fine pubescence.
   First year stem color.—young stem: 144C older stems: 144A (The Royal Horticultural Society Colour Chart), First year stem diameter is 4.5 cm. First year trunk diameter is 7.5 cm at the soil line.
   Stem Strength.—lateral stems are strong but benefit from being staked during flowering.
   Internodes' length.—6 cm in the lower half and 4 cm in the upper half.
Foliage description:
   Branch strength.—strong.
   Branch description.—branches are short, densely branched with short, broad leaflets.
   Leaf arrangement.—palmately compound (digitate) leaves with 7 leaflets on big fan leaves and 5 on small fan leaves per leaf.
   Texture (upper and lower surfaces).—Upper surface scabrid with non-visible stiff hairs; lower surface more or less densely pubescent, covered with sessile glands.
   Venation pattern.—Palmately Compound (Digitate), with serrated leaflets. Each serration has a lateral vein extending to its tip from the central (primary) vein of the leaflet. From each lateral vein there is usually a single spur vein (sublateral vein) extending to the notch of each serration.
   Color of emerging foliage (upper surface).—A cross between 144A and 144C (The Royal Horticultural Society Colour Chart, 1995 Edition.)
   Petiole length.—Average length of petiole of fan leaves 7 cm. More than 30% of the petioles are purple.
Vegetative bud description: Dried flower buds are deep dark green and highly resinous. Fragrance is weaker than similar varieties, having an earthy medicinal berry smell.
Flower description:
   Inflorescence is cone.—or head-like, dense cluster of false spikes with solitary flowers instead of cymes. Enclosed by glandular, beaked bracteole. Calyxes are densely covered with trichomes; Flowers are often paired. Petals are absent.
   Spike length.—ranges between 5 cm and 3 cm.
   Pistils description.—white, few, short, brown at maturation.
   Dried flower colour.—Dried flower buds are a deep, dark green.
   Flower size.—about 1 cm in diameter and about 1.1 cm in height.
   Flowering time.—8 weeks.
Peduncles:
   Strength.—Strong, but can bend horizontally from weight of flower buds.
   Texture.—Moderately smooth, glabrous.
   Pedicels description.—Short, scabrid with sessile glands and visible hairs.

Seeds may be acquired through STS breeding. This involves inducing a female plant to become a male plant by altering its hormones using (in this case) silver nitrate and sodium thiosulfate. The male plant may then be used to pollinate a female of the same variety and collect the seeds. The relative proportion of male plants is low/medium.

The plant variety is similar to the Erez, also a Cannabis indica subspecies.

The inflorescence contains a high amount of Cannabidiol (CBD) (16.3%) and a very low amount of THC (0.8%). This enables patients to obtain the medicinal benefits of CBD, without the psychoactive effects of THC.

This plant has strong anti-inflammatory properties. It can be used to treat a wide range of health disorders and diseases involving inflammation, as well as sleep disorders and digestive orders.

Prefers ‘soft water’; consider filtering the tap water if it has an EC higher than 0.6

Light was provided at a power of 600W HPS; plant distance from light was 100-120 cm.

| USDA Plants Growth Habit Code | FB |
|---|---|
| Vigor | 4.5/5 |
| Productivity | Good—500g/m2 indoor, |
| | 250-400g/plant outdoor |
| Male flowering timing | average |
| Flowering score | 7.5 |
| Branches | strong |
| Fruit | bloated |
| THC level | 0.8% |
| CBD level | 16.3% |

A crop was grown having the following times of growth cycle operations:

| | |
|---|---|
| date of cutting/or from seeding | 21 Apr. 2011 |
| date transferred to cones pot of 0.8 liter | 27 Apr. 2011 |
| Date of moved to pot of 11 liters | 23 May 2011 |
| Date of taking clones | 03 Jun. 2011 |
| Date—start to flower | 27 Jun. 2011 |
| Ending flowering | 23 Aug. 2011 |
| wet flowers (grams) | 524 |
| wet leaves (grams) | 250 |
| wet branches (grams) | 230.2 |
| dry flowers (grams) | 132.6 |
| Mothers | 1-1.5 meter |
| Pot size | 25 L |

Permits are required for growth of medical marijuana in certain states in the US and Israel.

1. A new and distinct Cannabis Sativa L. ssp. indica plant named Avidekel, characterized by a high amount of Cannabidiol of greater than approximately 16% and a very low amount of THC of less than 1%, as illustrated and described herein.
